SEQUENCE LISTING (10 sequences)

SEQ ID NO: 1 - DNA, 18 bp, Artificial Sequence
  Chemically synthesized
aacggctccggcatgtgc

SEQ ID NO: 2 - DNA, 18 bp, Artificial Sequence
  Chemically synthesized
gggcaggggtgttgaagg

SEQ ID NO: 3 - DNA, 18 bp, Artificial Sequence
  Chemically synthesized
gctggccaactatgcctc

SEQ ID NO: 4 - DNA, 19 bp, Artificial Sequence
  Chemically synthesized
gaaacagactgggccaatg

SEQ ID NO: 5 - DNA, 18 bp, Artificial Sequence
  Chemically synthesized
tgcctacctggacgaagc

SEQ ID NO: 6 - DNA, 18 bp, Artificial Sequence
  Chemically synthesized
cccagttcaggctcttag

SEQ ID NO: 7 - DNA, 18 bp, Artificial Sequence
  Chemically synthesized
cccaacgccatcttcaag

SEQ ID NO: 8 - DNA, 18 bp, Artificial Sequence
  Chemically synthesized
cttggacatccacacgtg

SEQ ID NO: 9 - DNA, 18 bp, Artificial Sequence
  Chemically synthesized
ctgttaccgccacttccc

SEQ ID NO: 10 - DNA, 18 bp, Artificial Sequence
  Chemically synthesized
ggtgcggtaccagtgcac

(19) United States
(12) Patent Application Publication
(10) Pub. No.: US 2009/0098177 A1
(43) Pub. Date: Apr. 16, 2009

(54) Methods and devices for tissue repair

(75) Inventors: Jerome Anthony Werkmeister, Victoria (AU); Wei-Bor Tsai, Taipei (TW); John Alan Maurice Ramshaw, Victoria (AU); Helmut Werner Thissen, Wheelers Hill (AU); Ken-Yuan Chang, Hsinchu (TW)

Correspondence Address: MCDERMOTT WILL & EMERY LLP, 600 13TH STREET, N.W., WASHINGTON, DC, 20005-3096, US

(73) Assignees: COMMONWEALTH SCIENTIFIC AND INDUSTRIAL RESEARCH INSTITUTE, Campbell (AU); INDUSTRIAL TECHNOLOGY RESEARCH INSTITUTE, Kuang Fu Road (TW)

(21) Appl. No.: 12/292,169

(22) Filed: Nov. 13, 2008

Related U.S. Application Data
(63) Continuation of application No. 10/470,946, filed on Dec. 15, 2003, now abandoned.

(30) Foreign Application Priority Data
Feb. 5, 2001 (AU) PR 2896

(51) Int. Cl. A61K 9/00; A61K 35/12

(52) U.S. Cl. 424/422; 424/93.21

(57) ABSTRACT

Methods for treating diseased or damaged tissue in a subject are disclosed, involving administering to said subject at a site wherein diseased or damaged tissue occurs, cells of a type(s) normally found in healthy tissue corresponding to the diseased or damaged tissue, and/or suitable progenitor cells thereof, in association with bioresorbable beads or particles and optionally a gel and/or gel-forming substance. Where the cells an/or suitable progenitor cells thereof are chondrocytes, embryonic stem cells and/or bone marrow stromal cells, the methods of the invention are suitable for treating, for example, articular cartilage degeneration associated with primary osteoarthritis. Also disclosed is a device having tissue-like characteristics for treating diseased or damaged tissue in a subject, wherein the device comprises cells of a type(s) normally found in healthy tissue corresponding to the diseased or damaged tissue, and/or suitable progenitor cells thereof, in association with bioresorbable beads or particles and optionally a gel and/or gel-forming substance.

### FIELD OF THE INVENTION

The present invention relates to methods and devices for treating diseased or damaged tissue, particularly articular cartilage degeneration associated with primary osteoarthritis, and other articular cartilage damage caused by, for example, sporting injuries or trauma. The present invention may also be applied to tissue augmentation (e.g. for cosmetic reasons).

### BACKGROUND OF THE INVENTION

Articular cartilage is found lining the bones within bone joints (e.g. the knee) where it allows for stable movement with low friction and provides resistance to compression and load distribution. The articular cartilage appears as a simple, avascular matrix of hyaline cartilage but, in fact, consists of a relatively complex formation of chondrocytes and extracellular matrix (ECM) organised into four zones (i.e. the superficial, transitional, middle and calcified zones) based upon matrix morphology and biochemistry. In turn, each of these zones consists of three distinct regions (i.e. the pericellular, territorial, and interterritorial regions). Chondrocytes, which comprise less than 5% of the volume of human articular cartilage, replace degraded ECM molecules and are thereby essential for maintaining tissue integrity (i.e. size and mechanical properties). The ECM includes a number of components including collagen (primarily, Type II collagen), glycoproteins, proteoglycans and tissue fluid which comprises up to about 80% of tissue weight of articular cartilage. The collagen component provides a fibre mesh structure to the ECM and the glycoproteins are thought to assist in the stability of the structure. The proteoglycans comprise large aggregating monomers (i.e. aggregans) which fill the inter-fibre spaces and, because of their ability to attract water, are believed to account for much of the resiliency and load distribution properties of articular cartilage. Finally, the tissue fluid, which includes a source of nutrients and oxygen, provides the articular cartilage with the ability to resist compression and return to its regular shape following deformation (for a review, see Temenoff and Mikos, 2000).

Joint pain resulting from articular cartilage degeneration or injury is a common condition which afflicts people of all ages. Its major causes are primary osteoarthritis and trauma causing loss of cartilage (Buckwalter and Mankin, 1998). Recently, it has been estimated that up to 43 million people in the United States of America alone suffer from some form of arthritis (see “Arthritis Brochure” at http://orthoinfo.aaos.org/), while cartilage damage arising from sporting injuries is also prevalent.

Unfortunately, and owing in part to its complex structure (Temenoff and Mikos, supra), articular cartilage has extremely little ability for self repair and, as a consequence, articular cartilage degeneration and injuries persist for many years and often lead to further degeneration (i.e. secondary osteoarthritis).

Treatment options for articular cartilage degeneration can be grouped according to four principles, i.e. replacement, relief, resection and restoration. Replacement of articular cartilage involves the use of a prosthesis or allograft. Relief of symptoms can be achieved by an osteotomy operation, which removes a portion of one of the bones in the defective joint so as to decrease loading and stress. Resection refers to surgical removal of the degenerated articular cartilage and subsequent uniting of the healthy, surrounding articular cartilage tissue. Such resection operations may or may not involve the use of interposition arthroplasty. Lastly, restoration refers to healing or regeneration of the joint surface, including the articular cartilage and the subchondral bone. This may involve an attempt to enhance self repair (e.g. through use of pharmaceutical agents such as growth factors, or subchondral drilling, abrasion or microfracture to “recruit” pluripotent stem cells from the bone marrow), or otherwise, regenerating a new joint surface by transplanting chondrocytes or other cells having the ability to regenerate articular cartilage.

Considerable research has been conducted in recent years on the development of suitable “restoration” treatments or, more specifically, treatments involving regeneration of a new joint surface (sometimes referred to as “biological resurfacing”). Such treatments may be less traumatic to a patient than an osteotomy or prosthetic replacement, and offer advantages over the use of allografts which may not be immunologically tolerated and which may contain foreign pathogens, or multiple autografts which, inevitably, cause damage at another site on the patient. One “biological resurfacing” treatment that has been proposed involves the harvesting of chondrocytes from an articular cartilage biopsy from the patient (Freed et al., 1999). These cells are expanded in culture, and administered back to the patient by injection under a periosteal flap, which is sutured to ensure that the expanded chondrocytes remain at the site requiring repair. While this treatment has shown considerable promise in human trials over the past decade (Temenoff and Mikos, supra), the need for a periosteal flap adds an additional restriction to the technique, and the act of sewing the periosteal flap over the injected chondrocytes can lead to damage to the adjacent tissue. Additionally, there is no evidence to suggest that the expanded cells remain phenotypically and functionally as chondrocytes; indeed, they may have de-differentiated into fibroblast-like cells that produce mechanically inferior tissue.

A potential alternative to the use of the above system of autologous cells and periosteal flap, is the use of preformed porous scaffolds that approximates the desired shape and form of the diseased or damaged tissue, and which have been seeded with chondrocytes and cultured for at least 2 to 3 weeks. The tissue equivalent that forms is then implanted at the required site (Thomson et al., 1995). Recent work with collagen-based scaffolds has been promising, however most of the current research being conducted in this area is concerned with identifying suitable synthetic polymer materials for scaffolds, since these may be produced in large amounts and should overcome the concerns surrounding the possibility of incomplete pathogen removal from donor collagen (Temenoff and Mikos supra). Particular examples of synthetic polymer materials being researched are fibres of FDA-approved polymers, poly(glycolide) (PGA), poly(lactide) (PLA) and copolymers poly(lactide-co-glycolide) (PLGA). These polymer fibres, which may be woven into a mesh, are biodegradable and therefore offer advantages over non-degradable polymers in that their gradual degradation steadily creates room for tissue growth and, secondly, they eliminate the need for surgical removal of the scaffold following restoration of the articular cartilage.

The use of scaffolds does, however, have the substantial disadvantage of necessitating surgery for implantation. Accordingly, other research groups have directed their efforts towards the development of polymers, which may be injected with chondrocytes and, subsequently, become cross-linked in situ to form a scaffold matrix. For example, fibrinogen and thrombin can be combined and injected wherein a degradable fibrin mesh is formed (Sims et al., 1998), and alginate has also been investigated since this may be cross-linked with calcium (Rodriguez and Vacanti, 1998). Alginate has, however, been found to be immunogenic (Kulseng et al., 1999) and invokes a greater inflammatory response than synthetic polymer materials (Cao et al., 1998). Thus, research has also been conducted with injectable synthetic polymer gel materials including copolymers of ethylene oxide and propylene oxide PEO-co-PPO (Cao et al., supra) and photopolymerizable end-capped block copolymers of poly(ethylene oxide) and an α-hydroxy acid (Hubbell, 1998).

The present invention relates to an alternative method for tissue regeneration, particularly articular cartilage regeneration, wherein chondrocytes and/or other suitable progenitor cells are bound to, or otherwise blended with, bioresorbable beads or particles for administration to a subject at a site where tissue regeneration is required. It is believed that the method avails itself of many of the advantages of biodegradable polymer scaffolds discussed above, including the ability to be administered by injection if desired. Additionally, and while not wishing to be bound by theory, it is thought that the use of beads or particles may provide mechanical and space-filling benefits while tissue regeneration is progressing by offering physical support and resistance to compression.

### DISCLOSURE OF THE INVENTION

Thus, in a first aspect, the present invention provides a method for treating diseased or damaged tissue in a subject, said method comprising administering to said subject at a site wherein said diseased or damaged tissue occurs, cells of a type(s) normally found in healthy tissue corresponding to said diseased or damaged tissue, and/or suitable progenitor cells thereof, in association with bioresorbable beads or particles and, optionally, a gel and/or gel-forming substance.

The said cells and/or progenitor cells may be associated with the beads or particles simply through mixing and may therefore not necessarily be bound to the beads or particles. The cells and/or progenitor cells may be mixed with the beads or particles by low shear agitation in a suitable vessel. The gel and/or gel-forming substance may be simultaneously mixed with the cells and/or progenitor cells and beads or particles, or alternatively mixed subsequently. However, preferably, the cells and/or progenitor cells are associated with the beads or particles by being bound thereto. This may be achieved by expanding the cells and/or progenitor cells in the presence of the beads or particles.

Thus, in a second aspect, the present invention provides a method for treating diseased or damaged tissue in a subject, said method comprising the steps of;

(i) obtaining cells of a type(s) normally found in healthy tissue corresponding to said diseased or damaged tissue and/or suitable progenitor cells thereof,
(ii) expanding said cells and/or progenitor cells in the presence of bioresorbable beads or particles whereby said expanded cells and/or progenitor cells become bound to the said beads or particles, and
(iii) administering to said subject the beads or particles with said cells and/or progenitor cells bound thereto, optionally in a gel and/or gel-forming substance, at a site wherein said diseased or damaged tissue occurs.

It will be appreciated by persons skilled in the art that between steps (i) and (ii) above, an additional expansion step(s) may be carried out. Such additional expansion step(s) may involve growth of the cells in, for example, monolayer(s).

It will also be appreciated by persons skilled in the art that it is not necessary to expand the cells and/or progenitor cells in the presence of the beads or particles at all and that, alternatively, the cells and/or progenitor cells could be expanded and, subsequently, bound to the beads or particles.

Thus, in a third aspect, the present invention provides a method for the treatment of diseased or damaged tissue in a subject, said method comprising the steps of;

(i) obtaining cells of a type(s) normally found in healthy tissue corresponding to said diseased or damaged tissue and/or suitable progenitor cells thereof,
(ii) expanding said cells and/or progenitor cells,
(iii) binding said expanded cells and/or progenitor cells to bioresorbable beads or particles, and
(iv) administering to said subject the beads or particles with said cells and/or progenitor cells bound thereto, optionally in a gel and/or gel-forming substance, at a site wherein said diseased or damaged tissue occurs.

The said cells and/or progenitor cells are selected such that they are of a type(s) suitable for regeneration of the particular diseased or damaged tissue type (e.g. mature differentiated cells of the tissue type to be treated). Thus, by way of example, for the treatment of diseased or damaged skin, the cells used in the methods of the present invention shall be fibroblasts and/or progenitor cells thereof. Where the tissue to be regenerated is bone, the cells shall be osteoblasts and/or progenitor cells thereof, while for the treatment of fatty tissues, the cells shall be adipocytes and/or progenitor cells thereof.

Preferably, the methods of the present invention are used for treating (e.g. repairing) articular cartilage degeneration or injury. In this regard, articular cartilage tissue regeneration may be achieved at the site of articular cartilage degeneration or injury, and the bioresorbable beads or particles are gradually degraded so that removal of the beads or particles following regeneration is not required. In this application of the methods of the present invention, the cells used are chondrocytes and/or progenitor cells thereof. Further, as mentioned above, it is thought that while tissue regeneration is progressing, the beads or particles provide mechanical and space-filling benefits. That is, they may provide a load-bearing cushion to the articular cartilage degeneration or injury by offering physical support to the bone joint, reduced friction during joint movement and resistance to compression. In addition, where the beads or particles are administered in a gel or gel-forming substance, the beads or particles appear to prevent gel contraction, which might otherwise adversely affect space-filling of the tissue defect.

The chondrocytes and/or progenitor cells may be harvested by any of the methods common to the art, but most conveniently, by tissue biopsy. Suitable chondrocyte progenitor cells are undifferentiated cells such as embryonic stem cells and bone marrow stromal cells. Preferably, the chondrocytes and/or progenitor cells are obtained from the subject to be treated.

The expansion step in the methods of the second and third aspects, preferably expand the cells and/or progenitor cells 5 to 2000-fold, more preferably, 10 to 100-fold, by any of the methods common to the art. For example, expansion may be achieved by cell culture in a suitable dish (such as a petri dish, with or without, for example, an agar gel being present), but more preferably, is conducted in a bioreactor where the culture medium is agitated and aerated. The expansion may, however, involve more than one stage. For example, chondrocytes and/or progenitor cells thereof may first be grown as a monolayer in a suitable dish, wherein cell spreading may be mediated by serum adhesion proteins such as fibronectin (Fn) and vitronectin (Vn), and subsequently grown in a bioreactor. As mentioned above, the expansion, or a portion of the expansion, may or may not be conducted in the presence of bioresorbable beads or particles. Also, when beads or particles are present during the expansion, or a portion of the expansion, the cells and/or progenitor cells may be removed and “re-seeded” onto bioresorbable beads or particles. In this case, the first mentioned beads or particles may not necessarily be bioresorbable beads or particles. Where the expansion involves culturing in a bioreactor, it is convenient to add bioresorbable beads or particles to the culture medium. However, where the expansion is conducted without beads or particles, it is necessary, as is clear from the above, to subsequently bind the expanded cells and/or progenitor cells to bioresorbable beads or particles.

A simple bioreactor that is suitable for expansion of cells (e.g. chondrocytes) and/or progenitor cells for use in the methods of second and third aspects, is a spinner flask. Alternatively, expansion of the cells and/or progenitor cells may be achieved with a tumbler-type bioreactor (eg: Synthecon™ Inc. STLV™ Rotary Cell Culture System) which may or may not be equipped with internal vanes to assist in movement of the cells, culture medium and bioresorbable beads or particles, if present.

Where chondrocytes are used, culturing in a spinner flask or tumbler-type bioreactor should ensure maintenance of cell phenotype. However, where the expansion involves culturing in an essentially still culture medium, it may be necessary to take steps to prevent de-differentiation of the chondrocytes. In both cases, the culture medium may include supplements, such as ascorbate or growth factors, which control the cell growth and characteristics.

The bioresorbable beads or particles utilised in the methods of the present invention are preferably sized such that they are readily injectable. Accordingly, the bioresorbable beads or particles preferably have a diameter or dimensions sized in the range of about 20 to 2500 μm, more preferably, with an average size of about 50 to 200 μm. Suitable bioresorbable beads may be of a regular shape (e.g. spheroid such as microspheres, ovoid, disc-like or rod-like) or a mixture of regular shapes. On the other hand, suitable bioresorbable particles will generally be comprised of a large variety of irregular shaped particles as would typically be produced from crushing or pulverising solid substances.

The bioresorbable beads or particles may be comprised of any pharmaceutically acceptable polymer including biologically-based polymers such as gelatin and collagen (especially type I and/or type II), and synthetic polymers such as those, which have been used in, cell scaffolds (i.e. PGA, PLA and PLGA), and mixtures of biologically-based and synthetic polymers. Alternatively, the bioresorbable beads or particles may be comprised of other pharmaceutically acceptable non-polymeric substances including bone particles (e.g. crushed bone and particles of demineralised bone). Also, the bioresorbable beads or particles may be comprised of a mixture of such polymers and non-polymeric substances.

Preferably, the bioresorbable beads or particles are of a size and density that allows thorough movement of the beads or particles in a spinner flask or tumbler-type bioreactor. This may assist in cell expansion and, where chondrocytes are being used, maintenance of chondrocyte phenotype.

The bioresorbable beads or particles may be functionalised or coated in a suitable material to enhance cell adherence (e.g. an antibody or fragment thereof which binds to a cell-surface antigen, or ECM proteins such as collagen Type I, II, VI, IX, XI, etc.) and/or, where chondrocytes are being used, may also be coated with an agent to assist in the maintenance of phenotype (e.g. a type II collagen). Additionally, the beads or particles may comprise other beneficial agents such as growth factors (e.g. TGFβ, EGF, FGF, IGF-1 and OP-1, etc.), glycosaminoglycans (GAGs) (e.g. aggrecan, decorin, biglycan, fibromodulin) and hydrophilic compounds (e.g. polylysine, chitosan, hyaluronan).

Preferably, the beads or particles, with suitable cells and/or progenitor cells associated therewith, are administered to a subject in a gel and/or gel-forming substance. However, additionally or alternatively, the beads or particles with suitable cells and/or progenitor cells associated therewith, may be administered in combination with a suitable pharmaceutically acceptable carrier (e.g. physiological saline, sterile tissue culture medium, etc.).

Suitable gel and/or gel-forming substances are preferably bioresorbable and of a type that ensures that the beads or particles are substantially retained at the site of administration. The gel and/or gel-forming substance may, therefore, comprise an adhesive material(s) (e.g. fibrin and/or collagen, or a transglutaminase system) to adhere the gel or formed gel to the tissues surrounding the site of administration. Alternatively, or additionally, the beads or particles may be substantially retained at the site of administration by entrapping the gel and/or gel-forming substance containing the beads or particles within tissue (e.g. the dermal and/or adipose tissue(s)) or under a tissue (e.g. a periosteal flap) or other membranous flap (e.g. a collagen membrane).

Suitable gels and gel-forming substances may comprise a biologically-based polymer (i.e. a natural or treated natural polymer) such as a collagen solution or fibrous suspension, hyaluronan or chitosan (hydrolysed chitin), or a synthetic polymer such as a photopolymerizable end-capped block copolymer of poly(ethylene oxide) and an α-hydroxy acid. The gel and/or gel-forming substance may also comprise other beneficial agents such as growth factors (including those mentioned above), glycosaminoglycans (GAGs) and hydrophilic compounds (such as those mentioned above).

In the methods of the second and third aspects, the cells and/or progenitor cells bound to the beads or particles, when ready for administration, may be confluent or sub-confluent. An average between about 3 and 500 cells and/or progenitor cells are preferably associated with each bioresorbable beads or particles. The numbers will, however, vary depending upon the characteristics (e.g. composition and size) of the beads or particles. For administration, it is preferred to use 1×10⁵ to 1×10⁹ cells and/or progenitor cells bound per 1 cm³ of beads or particles.

Where chondrocytes are used, the chondrocytes bound to the beads or particles may be administered to the subject, before or after the chondrocytes have commenced secreting extracellular matrix. The latter is, however, less preferred since the extracellular matrix can lead to the formation of aggregates, which may not be readily injectable.

In the method of the third aspect of the present invention, the cells and/or progenitor cells are first expanded and then (i.e. subsequently), bound to bioresorbable beads or particles. This may be achieved in a suitable dish (e.g. a petri dish) or in tissue culture flasks. Again, the bioresorbable beads or particles may be functionalised or coated in a suitable material to enhance cell adherence, and/or coated with an agent to assist in the maintenance of chondrocyte phenotype. The beads or particles may also comprise other beneficial agents such as growth factors, glycosaminoglycans (GAGs) and hydrophilic compounds.

In the method of the third aspect of the invention, the beads or particles with bound cells and/or progenitor cells can be administered to the patient immediately after step (iii), or after further culturing of the cells and/or progenitor cells on the beads or particles.

The administration of the cells and/or progenitor cells in association with the beads or particles and gel and/or gel-forming substance is preferably by injection or arthroscopic delivery.

The methods of the present invention are primarily intended for human use, particularly in relation to treatment of articular cartilage tissue degeneration or injury (e.g. in the knee, fingers, hip or other joints). However, it is also anticipated that the methods may well be suitable for veterinary applications (e.g. in the treatment of articular cartilage degeneration or injury in race horses, and in the treatment of articular cartilage degeneration or injury in companion animals).

The present invention also contemplates the production of a tissue-like device that may be surgically implanted into a subject for the treatment of diseased or damaged tissue.

Thus, in a fourth aspect, the present invention provides a device having tissue-like characteristics for treating diseased or damaged tissue in a subject, wherein said device comprises cells of a type(s) normally found in healthy tissue corresponding to said diseased or damaged tissue, and/or suitable progenitor cells thereof, in association with bioresorbable beads or particles and optionally a gel and/or gel-forming substance.

The device may be prepared by culturing said cells and/or progenitor cells in association with bioresorbable beads or particles and optionally a gel and/or gel-forming substance, for a period of time sufficient so as to form a tissue-like mass. The cells and/or progenitor cells may or may not be bound to the bioresorbable beads or particles. The bioresorbable beads may have fully degraded prior to implantation of the device, but preferably, the beads or particles are substantially intact within the device at the time of implantation.

In a fifth aspect, the present invention provides a method for treating diseased or damaged tissue in a subject, said method comprising implanting into said subject at a site wherein said diseased or damaged tissue occurs, a device according to the fourth aspect.

It will be readily appreciated by persons skilled in the art that a combination of different types of cells, potentially on the same or different types of beads, could be used to effect repair of the diseased or damaged tissue.

It will also be readily appreciated by persons skilled in the art that the present invention may be applied to tissue augmentation (e.g. treatment of scars or facial wrinkles).

By the term “bound” we refer to any mechanism by which cells and/or progenitor cells may adhere to a bioresorbable bead or particle so that substantially all of said cells and/or progenitor cells bound to a particular bioresorbable bead or particle remain bound to that bead or particle. Such mechanisms include binding of chondrocytes and/or progenitor cells to said bead via an antibody (which may be covalently bound to the bead), or via an ECM protein (eg. collagen Type I, II, VI, IX, XI, etc.), or fragments thereof, which may also be covalently bound to the bead.

By the term “gel” we refer to any viscous or semi-solid solution or suspension which is capable of retarding settling of bioresorbable beads or particles as described above (c.f. bioresorbable beads or particles will readily settle out of physiological saline). Such solutions and suspensions preferably do not flow through a #2 Zahn Cup (Gardco, Inc.) (44 ml placed in the #2 Zahn Cup) at 37° C. and atmospheric pressure in less than 30 seconds. More preferably, such solutions or suspensions do not flow through a #4 Zahn Cup (Gardco, Inc.), that is less than 5% of the initial volume (44 ml placed in the #4 Zahn Cup) flows through after 2 minutes at 37° C. and atmospheric pressure.

The terms “comprise”, “comprises” and “comprising” as used throughout the specification are intended to refer to the inclusion of a stated step, component or feature or group of steps, components or features with or without the inclusion of a further step, component or feature or group of steps, components or features.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

The invention is hereinafter further described by way of the following non-limiting examples and accompanying figures.

### BRIEF DESCRIPTION OF THE ACCOMPANYING FIGURES

FIG. 1 provides microscopy images of chondrocyte cell growth on gelatin beads (A) and PLGA beads (B) (Examples 8 and 10).

FIG. 2 shows results of evaluation of cells for phenotype using RT-PCR, wherein PCR products are analysed by electrophoresis on 2% agarose gels (Example 20).

FIG. 3 shows the effect of beads on gel contraction after a 2-week culture of chondrocytes with and without beads (gelatin) in a collagen type I gel (Example 28).

FIG. 4 shows an example of new tissue formation using cultured chondrocytes on demineralised bone particles with a collagen type I gel (Example 31).

### EXAMPLE 1

### Chondrocyte Isolation

Fresh cartilage tissue is collected in DMEM/10% FBS or autologous serum containing 100 μg/ml penicillin and streptomycin. After weighing, the tissue is placed in a sterile petri dish containing 3-4 ml of DMEM and dissected into 1 mm³ pieces using a sharp sterile scalpel. It is then digested with 10% w/v trypsin in PBS at 37° C. for 1 hour. Approximately 2 ml of 10% w/v trypsin is used per gram of tissue. The residual tissue pieces are collected by centrifugation (1000 rpm, 5 mins) and washed with PBS, then water (using approximately 5-10 ml per gram of tissue). A second digestion step is then performed overnight at 37° C. using 2 ml of a mixture of bacterial collagenase and hyaluronidase per gram of tissue. The digestion mixture is prepared by adding 2 mg hyaluronidase (1520 units) and 200 μl of collagenase stock (taken from a 3000 unit/ml stock, stored at −70° C. in a buffer of 50 mM tris, 10 mM CaCl₂, pH 7.0) to 2 ml of DMEM and filter sterilising. The digested tissue is passed through a 70 μm Nylon cell strainer and the cells are washed and collected by centrifugation. Cell numbers and viability are assessed using a trypan blue count on a small known aliquot.

### EXAMPLE 2

### Fibroblast Isolation

Fresh skin, after hair removal and washing in 70% ethanol, is collected in DMEM/10% FBS or autologous serum containing 100 μg/ml penicillin and streptomycin. The tissue is placed in a sterile petri dish containing 3-4 ml of DMEM and dissected into 1 mm³ pieces using a sharp sterile scalpel. The tissue pieces are left in culture in DMEM/10% FBS or autologous serum containing 100 μg/ml penicillin and streptomycin to allow migration of fibroblasts onto the tissue culture plastic. After cells are visible on the tissue culture plastic, the tissue is removed and the cells sub-cultured. Cell numbers and viability are assessed using a trypan blue count on a small known aliquot.

### EXAMPLE 3

### Osteoblast Isolation

Fresh cortical bone is collected in DMEM/10% FBS or autologous serum containing 100 μg/ml penicillin and streptomycin. The bone is placed in a sterile petri dish containing 3-4 ml of DMEM. The bone piece(s) are left in culture in DMEM/10% FBS or autologous serum containing 100 μg/ml penicillin and streptomycin to allow migration of osteoblasts onto the tissue culture plastic. After cells are visible on the tissue culture plastic, the bone is removed and the cells sub-cultured. Cell numbers and viability are assessed using a trypan blue count on a small known aliquot.

### EXAMPLE 4

### Stem Cell Isolation

Adult mesenchymal stem cells (MSC) are harvested from bone marrow aspirates. The marrow is washed twice with sterile PBS then resuspended in DMEM/10% FBS or autologous serum containing 100 μg/ml penicillin and streptomycin. Marrow cells are then layered onto a Percoll cushion (1.073 g/ml density) and cells collected after centrifugation for 30 min. at 250 g and transferred to tissue culture flasks. Various additives including dexamethasone, growth factors and cytokines are used to select and propagate specific cell lineages.

### EXAMPLE 5

### Cell Culture in Monolayers

Cells, such as fibroblasts, chondrocytes, osteoblasts and other types isolated according to the protocols described above in Examples 1-4, are cultured on tissue culture plastic in DMEM/10% FBS or autologous serum containing 100 μg/ml penicillin and streptomycin, at 37° C. in 5% carbon dioxide atmosphere. Medium additions or change is performed every 2 days. Cells are grown to confluency, then trypsinised and replated into flasks as monolayers or transferred to beads/particles.

### EXAMPLE 6

### Cell Culture on Non-Resorbable Beads

Beads or particles, for example Cytodex beads (Pharmacia Biotech), providing a surface area of 250-500 cm², are pre-washed with 50 ml of warmed media (DMEM/10% FBS or autologous serum containing 100 μg/ml penicillin and streptomycin) at 37° C. then placed inside a 125 ml spinner bottle. 1×10⁵ cells, either freshly isolated cells, previously passaged cells or previously isolated and frozen cells, are added to the beads or particles. The bottle is then stirred in a 37° C. incubator (with 5% CO₂), at 25 rpm intermittently for 2 minutes every 30 minutes for 3 hours, then intermittently for 2 minutes every 30 minutes for the next 3 hours, then continuously first at 45 rpm for 15 minutes, then 50 rpm for 15 minutes, 55 rpm for 15 minutes, then to the final speed of 60 rpm. The cells are then grown at this speed until 90% confluence is achieved, usually 5-8 days depending on the original inoculum. For collection of the cells on the beads or particles, either for release and further seeding or for preparation for delivery to a patient or further processing, the cells and beads are washed with warm, 37° C. PBS and collected by centrifugation.

### EXAMPLE 7

### Preparation of Gelatin Beads

Gelatin microparticles are synthesized by using emulsion method. Briefly, gelatin is dissolved in 50 mM acetic acid to 20% (w/v). Two hundred milliliters olive oil is warmed up to 37° C. The warmed olive oil is stirred at 300 rpm. Forty millilitres gelatin solution kept at 37° C. is then applied to olive oil through a 20-gauge needle. This solution is also prepared containing 10% w/w native collagen. The emulsion is kept stirred for 90 minutes. The emulsion is then cooled down by stirring at 4° C. for 30 minutes in order to harden the gelatin particles. Five hundred millilitres of 0.2% Triton X-100 in PBS is added to the emulsion and stirred at room temperature for 10 minutes. The mixture is then put in a separating funnel and settled for one hour. The liquid in the lower portion is collected and after gelatin microparticles precipitate, the upper liquid decanted off carefully and the particles rinsed with water two times. Five hundred millilitres of 0.1% glutaraldehyde in PBS is added to the gelatin microparticles and stirred for one hour for cross-linking. The cross-linked gelatin beads are then rinsed with water several times and soaked in ethanol. The ethanol is decanted and the gelatin microparticles dried under vacuum. Before seeding cells, the gelatin beads are rehydrated with PBS overnight and then with chondrocyte medium. The average size of gelatin microparticles is about 110 μm.

### EXAMPLE 8

### Cell-Culture on Gelatin Beads

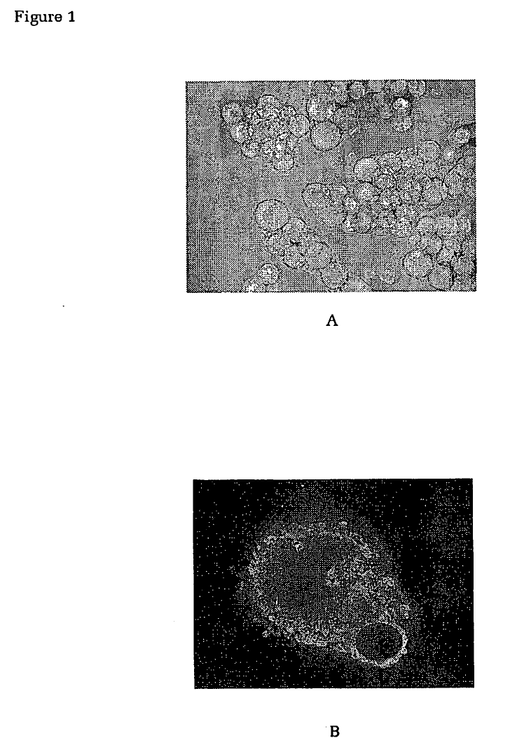

Gelatin beads, providing a surface area of 250-500 cm², are pre-washed with 50 ml of warmed media (DMEM/10% FBS or autologous serum containing 100 μg/ml penicillin and streptomycin) at 37° C. then placed inside a 125 ml spinner bottle. 1×10⁵ cells, either freshly isolated cells, previously passaged cells or previously isolated and frozen cells, are added to the beads or particles. The bottle is then stirred in a 37° C. incubator (with 5% CO₂), at 25 rpm intermittently for 2 minutes every 30 minutes for 3 hours, then 45 rpm intermittently for 2 minutes every 30 minutes for the next 3 hours, then continuously first at 45 rpm for 15 minutes, then 50 rpm for 15 minutes, 55 rpm for 15 minutes, then to the final speed of 60 rpm. The cells are then grown at this speed until 90% confluence is achieved, usually 5-8 days depending on the original inoculum. For collection of the cells on the beads or particles, either for release and further seeding or for preparation for delivery to a patient or further processing, the cells and beads are washed with warm, 37° C. PBS and collected by centrifugation. FIG. 1A shows cell growth on gelatin beads 7 days after addition of chondrocytes to the gelatin beads.

### EXAMPLE 9

### Preparation of PLGA Beads and Particles

Poly(lactide-co-glycolide) 85:15 w/w (PLGA) was dissolved in tetrahydrofuran and then emulsified into an aqueous solution containing 1% polyvinylalcohol by stirring. PLGA beads were collected by allowing them to settle, and were washed 5 times with water by decantation. Beads were then dried in a vacuum over night. Beads in the range of 30 μm to 300 μm were typically obtained, with an average size of 105 μm. Beads were fractionated into a narrower size range, 80 μm to 120 μm, by sieving. Alternatively, PLGA particles in the desired size range were obtained by crushing larger particles in a homogeniser, using a suspension of 1 g PLGA in 500 ml of water. Sieving provided particles of irregular shape in the desired size range, for example 50 μm to 250 μm. Surface modification of the PLGA beads and particles was carried out by adsorption of collagen I or collagen II from a solution containing 50 μg/ml collagen in phosphate buffered saline at room temperature for 1 hour. Subsequent washing in phosphate buffered saline removed loosely bound collagen.

### EXAMPLE 10

### Cell Culture on PLGA Beads

PLGA beads providing a surface area of 250-500 cm², are pre-washed with 50 ml of warmed media (DMEM/10% FBS or autologous serum containing 100 μg/ml penicillin and streptomycin) at 37° C. then placed inside a 125 ml spinner bottle. 1×10⁵ cells, either freshly isolated cells, previously passaged cells or previously isolated and frozen cells, are added to the beads or particles. The bottle is then stirred in a 37° C. incubator (with 5% CO₂), at 25 rpm intermittently for 2 minutes every 30 minutes for 3 hours, then 45 rpm intermittently for 2 minutes every 30 minutes for the next 3 hours, then continuously first at 45 rpm for 15 minutes, then 50 rpm for 15 minutes, 55 rpm for 15 minutes, then to the final speed of 60 rpm. The cells are then grown at this speed until 90% confluence is achieved, usually 5-8 days depending on the original inoculum. For collection of the cells on the beads or particles, either for release and further seeding or for preparation for delivery to a patient or further processing, the cells and beads are washed with warm, 37° C. PBS and collected by centrifugation. FIG. 1B shows chondrocyte culture on PLGA beads 14 days after chondrocytes were added to the PLGA beads. The chondrocytes have been stained with goat anti-type II collagen antibodies thereby indicating type II collagen synthesis.

### EXAMPLE 11

### Preparation of Bone Particles

Fresh bone, free from adherent tissue and rinsed with phosphate buffered saline (PBS) is dried and then crushed and milled to provide particles which are separated by sieving, to give for example a fraction that passes through a 120 micron sieve, but is retained by an 80 micron sieve. These particles are degreased by washing in methanol, dichloromethane and acetone. Particles are then washed in 2 changes of PBS and then water and dried. Demineralised bone particles are prepared by agitation of bone particles in 0.5 M EDTA, pH 7.4, for 20 hr. After separation by gentle centrifugation, this process was repeated at least a further two times.

### EXAMPLE 12

### Cell Culture on Bone Particles

Culture of cells on bone particles was as in Example 10, except bone particles, both untreated and demineralised, are used instead of PLGA beads.

### EXAMPLE 13

### Cell Culture in a Bioreactor

Beads or particles with cells attached, as described in Examples 6 or 8 or 10 or 12, are placed in a bioreactor, such as a High Aspect Ratio Vessel of a Synthecon™ Rotary Cell Culture System, where the vessel is filled with DMEM/10% FBS or autologous serum containing 100 μg/ml penicillin and streptomycin and air bubbles removed. Culture is continued in a humidified incubator with 5% carbon dioxide present, with the initial rotation speed at 15 rpm. The speed is then further adjusted, dependent on the nature and size of the bead or particle so that the beads or particles are not settling nor colliding with the edge of the vessel, but are forming a fluid orbit within the culture vessel. Medium change or addition is every 1 or 2 days.

### EXAMPLE 14

### Removal and Transfer of Cells from a Monolayer Culture

Warm, 37° C., 0.3% w/v trypsin in PBS is added directly to tissue culture flask, 5 ml per 25 cm². After standing for up to 5 minutes, cells are dislodged from the plastic by gentle pipette action or by gentle mechanical action. Cells in the trypsin solution are collected by centrifugation at 1000 rpm for 5 mins. The supernatant is then removed and the cells gently resuspended in 5 ml of media. Cells are counted using a trypan blue method.

### EXAMPLE 15

### Removal of Cells from Polymer Beads

Apply 6 ml of warm 0.3% w/v trypsin directly to the collected and washed cells on beads and incubate at 37° C. for 10 to 15 minutes without stirring. Apply 20 ml of warm PBS to the mixture and gently pipette up and down to dislodge cells from beads or particles, which have a size greater than 70 μm. Transfer cells and beads or particles through a 70 μm filter into a 50 ml tube. Collect the cells that pass through the filter by centrifugation at 1000 rpm for 5 mins. Remove the supernatant and gently resuspend the cells in 5 ml of media. Cells are counted using a trypan blue method.

### EXAMPLE 16

### Removal of Cells from Gelatin Beads

Apply 6 ml of warm 0.3% w/v trypsin directly to the collected and washed cells on beads and incubate at 37° C. for 20 minutes. The gelatin beads were digested by the enzyme, releasing the cells into solution without the need for extensive mechanical agitation. Cells were collected by centrifugation at 1000 rpm for 5 mins. Remove the supernatant and gently resuspend the cells in 5 ml of media. Cells are counted using a trypan blue method.

### EXAMPLE 17

### Transfer of Cells onto Resorbable Beads for Implant

Cells, such as fibroblasts, chondrocytes, osteoblasts or other types, either freshly isolated, or previously passaged in monolayer culture or on non-resorbable beads or particles or on resorbable beads or particles, or previously isolated, cultured and frozen, are suspended in warmed media (DMEM/10% FBS or autologous serum containing 100 μg/ml penicillin and streptomycin) at 37° C., and added to pre-washed beads or particles, as in Examples 7 or 9 or 11, and attachment is by a gradual increase in agitation, as in Examples 6 or 8 or 10 or 12.

### EXAMPLE 18

### Evaluation of Cells by Alcian Blue Staining

An advantage of culturing cells on beads or particles (Example 6, 8, 10, 12) is the control of phenotype. For articular cartilage, the phenotype is monitored using a variety of histochemical and immunohistochemical markers that can distinguish chondrocytes from de-differentiated fibrochondrocytes. Alcian blue, a general stain for the glycosaminoglycans of articular cartilage, is prepared as a 2% filtered solution in 3% acetic acid at pH 2.5. After fixing in neutral buffered formaldehyde for 2-3 min, slides are incubated in 3% acetic acid for 3 min. Alcian blue solution is applied for at least 20 hr at 37° C., slides are rinsed with water and a 2 minute neutral red stain is applied. An ethanol rinse is used prior to mounting in Histoclear.

### EXAMPLE 19

### Evaluation of Cells by Immunohistological Staining

The phenotype of cultured cells is monitored by specific immunological markers. For articular chondrocytes antibodies against collagen type II is used to monitor the correct phenotype and an anti-collagen type I antibody is used to monitor the extent of change or de-differentiation. If cells are to be stained for matrix production, for example by anti-collagen antibodies, fresh ascorbic acid must be added to cultures daily to a final concentration of 50 μg/ml for at least 6 days. After washing in warm PBS, cells on beads are pre-fixed, once in 50% (v/v) methanol in PBS for 10 minutes, twice in cool 70% (v/v) methanol in PBS for 10 minutes, then finally in 70% (v/v) ethanol in H₂O. Formalin or glutaraldehyde may be used as alternative fixatives for use with proteoglycans stains such as Alcian Blue. The primary antibody is diluted in PBS (e.g. goat anti type II collagen diluted 1 in 5 with PBS) and is applied for 1 hr at room temperature, then, after washing with PBS, an FITC-conjugated antibody diluted in PBS (e.g. rabbit anti goat FITC diluted 1 in 200 with PBS) is applied for 1 hr at room temperature. After washing with PBS twice, the beads are resuspended in mounting medium (e.g. 90% glycerol, 10% PBS, 0.025% DABCO). Fluorescent images are collected on an Optiscan confocal microscope.

### EXAMPLE 20

### Evaluation of Cells by In Situ Hybridisation and RT-PCR

Cells for in situ hybridisation characterisation are fixed as in Example 19. In situ-hybridization for mRNA encoding, for example collagen type I or collagen type II is performed using UTP-³³P detection following the method of Bisucci T, Hewitson T D, Darby I A, (2000) “cRNA probes: comparison of isotopic and non-isotopic detection methods”, in Methods in Molecular Biology, 123: 291-303. A type I collagen riboprobe consisting of 372 bp region of the human collagen pro α1(I) gene or a type II collagen riboprobe consisting of a 200 bp region of the bovine collagen α1(II) gene, is used.

For RT-PCR cells (pig chondrocytes) are cultured in monolayers and retrieved as in Example 5 and Example 14. Cells are lysed thoroughly in 1 ml REzol™ C&T (USA) by vortexing. The cell lysate is transferred to a microfuge tube, and incubated for 5 minutes at room temperature. Cell lysate is then mixed vigorously with 0.2 ml of chloroform and incubated at room temperature for 2 minutes. After centrifugation at 12,000×g for 15 minutes at 4° C., the upper aqueous layer is transferred to a new microfuge, and an equal volume of isopropanol is added and mixed gently. The samples were incubated at room temperature for 10 minutes and centrifuged at 12,000×g for 10 minutes at 4° C. The supernatant is removed carefully, and the RNA pellet is washed in 1 ml of 75% ethanol by vortex mixing and then centrifuged at 12,000×g for 5 minutes at 4° C. The ethanol is then removed carefully and the RNA pellet dried by air. The RNA pellet is dissolved in 20 μl of DEPC-treated water. The mRNA is then reverse-transcribed into cDNA by using oligo-dT primer and SUPERSCRIPT™II following manufacturer's recommendations (Life Technologies).

Aliquots of 2 μl from the RT reactions are used for amplification of transcripts using primers specific for the analyzed genes. PCR reactions are carried out by 3 minutes denaturation at 95° C., followed by 35 cycles of 1 minute denaturation at 95° C., 1 minute annealing at 50° C. and 1 minute elongation at 72° C. The primers for analyzed genes are designed as following:

| | |
|---|---|
| β-actin: | |
| 5′-AACGGCTCCGGCATGTGC-3′ | (SEQ ID NO:1) |
| and | |
| 5′-GGGCAGGGGTGTTGAAGG-3′ | (SEQ ID NO:2) |
| Type I collagen: | |
| 5′-GCTGGCCAACTATGCCTC-3′ | (SEQ ID NO:3) |
| and | |
| 5′-GAAACAGACTGGGCCAATG-3′ | (SEQ ID NO:4) |
| Type II collagen: | |
| 5′-TGCCTACCTGGACGAAGC-3′ | (SEQ ID NO:5) |
| and | |
| 5′-CCCAGTTCAGGCTCTTAG-3′ | (SEQ ID NO:6) |
| SOX9: | |
| 5′-CCCAACGCCATCTTCAAG-3′ | (SEQ ID NO:7) |
| and | |
| 5′-CTTGGACATCCACACGTG-3′ | (SEQ ID NO:8) |
| Aggrecan: | |
| 5′-CTGTTACCGCCACTTCCC-3′ | (SEQ ID NO:9) |
| and | |
| 5′-GGTGCGGTACCAGTGCAC-3′ | (SEQ ID NO:10) |

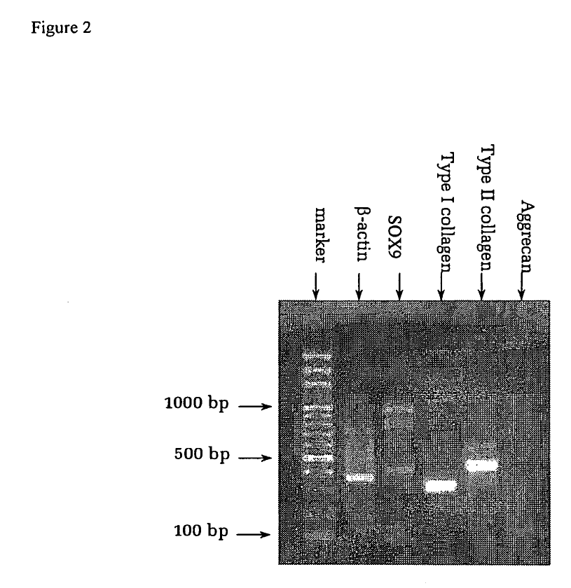

This is shown in FIG. 2.

### EXAMPLE 21

### Synthetic Gel Preparation

A suitable gel, that is bioresorbable, is formed by using a precursor consisting of PEO polymerised at its termini with oligomers of α-hydroxy acids, such as glycolic acid or lactic acid, and end capped at all oligo(α-hydroxy acid) termini with a polymerisable acrylate group, allowing polymerisation of the precursor to form a gel by brief exposure to long wavelength ultraviolet light.

### EXAMPLE 22

### Preparation of a Cells and Beads and Synthetic Gel Mixture

Cells, after removal from a gelatin bead substrate as shown in Example 8, or from other substrates, are mixed with fresh gelatin beads, made as in Example 7, or other bioresorbable beads or particles as in Example 9 or Example 11, in DMEM containing autologous serum or bovine fetal calf serum, and mixed with a synthetic gel precursor, such as that of Example 21, to form a uniform mixture, with the gel being formed by a brief exposure to ultraviolet light.

### EXAMPLE 23

### Biological (Collagen) Gel Preparation

Four grams of type I collagen, type II collagen, or mixtures of these collagens were dissolved in 1 litre 50 mM acetic acid solution. The collagen solution was spun at 9500 rpm, 4° C. for 45 minutes. The supernatant was collected. The collagen solution was put into a dialysis bag and then dialyzed against 25 litres IM acetic acid for two days, then against 25 litres water for four days with multiple water changes. The collagen solution was then concentrated in the sealed dialysis bag by hanging in a laminar flow hood for a day. The final concentration of the collagen solution was about 20 mg/ml (2% w/v).

### EXAMPLE 24

### Preparation of a Cells, Beads and Biological Gel Mixture

Cells, after removal from a gelatin bead substrate as shown in Example 8, or from other substrates, are mixed with fresh gelatin beads, made as in Example 7, or other bioresorbable beads or particles, in DMEM containing autologous serum or bovine fetal calf serum, and mixed with a biological gel or precursor, such as a 2% collagen solution prepared as in Example 23, to form a uniform mixture with the cells and beads or particles uniformly mixed, with gel formation being achieved by incubation of the mixture at 37° C.

### EXAMPLE 25

### Preparation of Cells-on-Beads and a Synthetic Gel Mixture

Cells attached to a gelatin bead substrate as shown in Example 8, or to other bioresorbable beads or particles, are collected by allowing the culture mixture to settle, with the excess culture media then being removed. The cells on the beads are then mixed with a synthetic gel precursor, such as that of Example 21, to form a uniform mixture, with the gel being formed by a brief exposure to ultraviolet light.

### EXAMPLE 26

### Preparation of Cells-on-Beads and a Biological Gel Mixture

Cells attached to a gelatin bead substrate as shown in Example 8, or to other bioresorbable beads or particles, are collected by allowing the culture mixture to settle, with the excess culture media then being removed. The cells on the beads are then mixed with a biological gel or precursor, such as a 2% collagen solution prepared as in Example 23, to form a uniform mixture. Nine parts of the collagen solution was mixed with one part of 10×DMEM and 0.1 part of 1N NaOH. Four parts of this mixture was mixed 1 part of chondrocyte-gelatin bead composites. Gel formation was achieved by incubation at 37° C. incubator for an hour, or could be achieved by body temperature for an implanted mixture.

### EXAMPLE 27

### In Vitro Culture of a Cells/Beads/Biological Gel Mixture

A biological gel containing cells and beads, as prepared in Example 24, is transferred, for example to a 24-well plate, and 1.5 ml of chondrocyte medium is added to each sample. Chondrocyte medium is changed every other day and 100 μg/ml of ascorbic acid is supplied every day. For in in vitro evaluation, samples are collected after 3 days, 7 days, 14 days, 21 days and 28 days.

### EXAMPLE 28

### In Vitro Culture of a Cell-on-Beads/Biological Gel Mixture

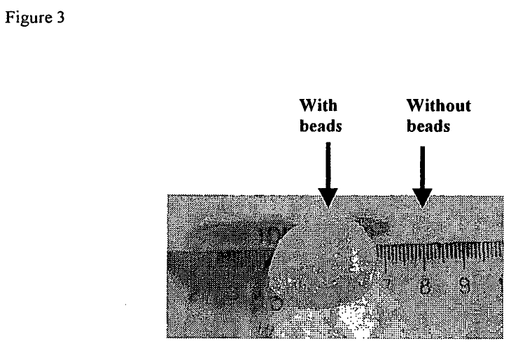

A biological gel containing cells-on-beads, as prepared in Example 26, is transferred to a cell culture plate and cultured in the presence of ascorbic acid as described in Example 27. Chondrocytes associated with the beads proliferate in the gel by day 3 and secreted new matrix of collagen type II and glycosaminoglycans consistent with the chondrocyte phenotype. The presence of the beads substantially reduces the rate and extent of gel contraction as shown in FIG. 3.

### EXAMPLE 29

### In Vitro Culture of a Cells/Beads/Synthetic Gel Mixture

A synthetic gel containing cells and beads, as prepared in Example 22, is transferred to a cell culture plate and cultured in the presence of ascorbic acid as described in Example 27.

### EXAMPLE 30

### In Vitro Culture of a Cells-on-Beads/Synthetic Gel Mixture

A synthetic gel containing cells on beads, as prepared in Example 25, is transferred to a cell culture plate and cultured in the presence of ascorbic acid as described in Example 27.

### EXAMPLE 31

### Implant of a Cells/Beads/Biological Gel Mixture into Animals

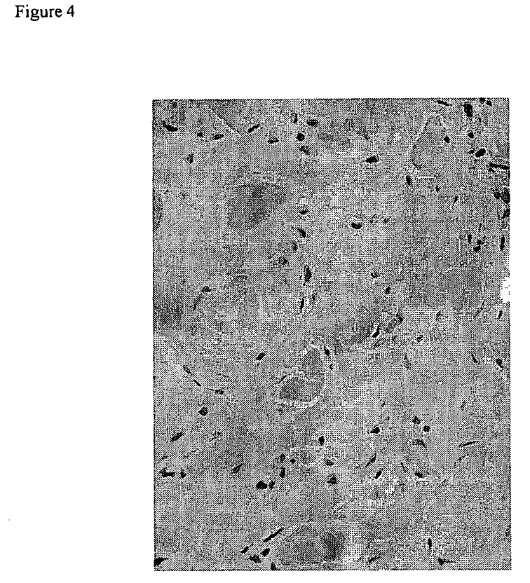

Either a cells and beads or a cells-on-beads in a type I collagen gel, as shown in Example 24 or 26, is injected subcutaneously into nude mice. Sacrifice of animals after 1 month and 2 months allows histological and immunohistological evaluation of the new tissue formed. Explants from nude mice show that articular cartilage can be produced using a variety of beads including gelatin, modified gelatin with collagen type I, and demineralised bone. Using type I collagen as the delivery gel, good tissue formation is noted within 1 month and continued at 2 months. Histochemical and immunohistochemical evaluation as described in Examples 18, 19 and 20 demonstrates the correct matrix and cartilage phenotype. FIG. 4 shows an example of new tissue formation using cultured chondrocytes on demineralised bone particles with a collagen type I gel.

### EXAMPLE 32

### Implant of an In Vitro Cultured Material into Animals

Either a cells and beads or a cells-on-beads in a biological gel mixture, for example using fibroblasts, chondrocytes or osteoblasts and gelatin beads in a type I collagen gel, as shown in Example 27 or 28 is surgically implanted subcutaneously into nude mice. Sacrifice of animals after 1 month and 2 months allows histological evaluation of the new tissue formed.

### EXAMPLE 33

### Implant of a Cells-on-Beads/Synthetic Gel Mixture into Animals

Either a cells and beads or a cells-on-beads in a synthetic gel mixture, for example a polyethylene glycol/lactic-glycolic acid/α-hydroxy acid type as shown in Example 22 or 25 is injected subcutaneously into nude mice. Sacrifice of animals after 1 month and 2 months allows histological evaluation of the new tissue formed.

### EXAMPLE 34

### Repair of a Cartilage Defect Using a Cell Containing Mixture

A preparation of cells (chondrocytes) and beads or particles and a gel is used. This mixture, for example chondrocytes attached to a gelatin bead substrate in a 2% type I collagen mixture, as shown in Example 26, is loaded into a syringe with a needle of sufficient diameter to allow easy passage of the beads or particles, such as 22 gauge. The material is then injected into a cartilage defect established in the knee of a sheep. The implanted material may also be retained in place by affixing a piece of autologous periosteum over the implanted chondrocyte containing material. After closure of the wound, the knee is kept temporally immobile to allow the collagen to form a semi-solid gel.

### EXAMPLE 35

### Repair of a Cartilage Defect Using a Cell Containing Mixture

Repair of a knee defect using a preparation of cells (chondrocytes) and beads or particles and a gel is achieved as shown in Example 34, except that a synthetic gel, as shown in Example 21 is used, with gel formation being achieved once the material is in the cartilage defect by brief exposure to ultraviolet light. The implanted material may also be retained in place by affixing a piece of autologous periosteum over the implanted chondrocyte containing material.

### EXAMPLE 36

### Repair of a Cartilage Defect Using an In Vitro Cultured Implant

A preparation of cells (chondrocytes) and beads or particles and a gel is used. This mixture, for example chondrocytes attached to a gelatin bead substrate in a 2% type I collagen mixture, as shown in Example 27, is held in cell culture supplemented by ascorbic acid for 10 days to allow a tissue like material to form containing the chondrocytes and gelatin beads. The tissue like material is then surgically implanted into a cartilage defect established in the knee of a sheep. The implanted material may also be retained in place by affixing a piece of autologous periosteum over the implanted chondrocyte containing material.

### EXAMPLE 37

### Repair of a Bone Defect Using a Cell Containing Mixture

A material is prepared as in Example 34, but with osteoblasts as the cell component and crushed bone particles, and is injected into a round defect in a sheep femur. Histological examination after 2 months is used to demonstrate bone repair.

### EXAMPLE 38

### Repair of a Bone Defect Using a Cell Containing Mixture

A material containing osteoblasts, crushed bone particles and type I collagen is prepared as in Example 37, but with the addition of BMP 2 or other growth factors. The material is injected into a round defect in a sheep femur and examined by histology after 2 months to demonstrate bone repair.

### EXAMPLE 39

### Repair of a Tissue Defect Using a Cell Containing Mixture

A material is prepared as in Example 34, but with fibroblasts as the cell component and gelatin beads, and is injected subcutaneously into sheep. Histological examination after 2 months is used to demonstrate tissue repair.

### EXAMPLE 40

### Repair of a Tissue Defect Using a Cell Containing Mixture

A material is prepared as in Example 34, but with adipocytes as the cell component and gelatin beads, and is injected subcutaneously into sheep. Histological examination after 2 months is used to demonstrate tissue repair.

### EXAMPLE 41

### Repair of a Tissue Defect Using a Cell Containing Mixture

A material is prepared with two cell types, fibroblasts and adipocytes, as the cell component, cultured separately on gelatin beads, as in Examples 39 and 40, which are mixed in the collagen gel, and injected subcutaneously into sheep. Histological examination after 2 months is used to demonstrate tissue repair.

### REFERENCES

Buckwalter, J. A., Mankin, H. J. Articular cartilage: degeneration and osteoarthritis, repair, regeneration and transplantation. AAOS Inst. Course Lect. 1998; 47: 487-504.
Cao Y., Rodriguez A., Vacanti M., Ibarra C., Arevalo C., Vacanti C. Comparative study of the use of poly(glycolic acid), calcium alginate and pluronics in the engineering of autologous porcine cartilage. J Biomater Sci Polym Edn, 1998; 9: 475-487.
Hubbell J. A., Synthetic biodegradable polymers for tissue engineering and drug delivery. Current Opinion in Solid State & Materials Science, 1998; 3: 246-251.
Kulseng B, Skjak-Braek G, Ryan L, Andersson A, King A, Faxvaag A, Espevik T. Transplantation of alginate microcapsules. Transplantation, 1999; 67: 978-984.
Freed, L. E., Martin, I., Vunjak-Novakovic, G. Frontiers in Tissue Engineering: In vitro Modulation of Chondrogenesis. Clinical Orthopaedics and Related Research, 1999; 3675: S46-S58.
Rodriguez, A. M., Vacanti, C. A. Tissue engineering of cartilage. In: Patrick Jr C. W., Mikos, A. G., McIntire L. V. editors. Frontiers in tissue engineering. New York: Elsevier Science, 1998; 400-411.
Sims, C. D., Butler P. E. M., Cao, Y. L., Casanova, R., Randolph, M. A., Black, A., Vacanti, C. A., Yaremchuk, M. J. Tissue engineered neocartilage using plasma derived polymer substrates and chondrocytes. Plast Reconstr. Surg, 1998; 101: 1580-1585.
Temenoff, J. S., Mikos, A. G. Review: tissue engineering for regeneration of articular cartilage. Biomaterials, 2000; 21: 431-440.
Thomson, R. C., Wake, M. C., Yaszemski, M. J., Mikos, A. G. Biodegradable polymer scaffolds to regenerate organs. Adv. Polym. Sci, 1995; 122: 245-274.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1-135. (canceled)

136. A method for treating diseased or damaged tissue in a subject, said method comprising administering by injection to said subject within or under a tissue at a site wherein said diseased or damaged tissue occurs, cells of a type(s) normally found in healthy tissue corresponding to said diseased or damaged tissue, and/or suitable progenitor cells thereof, in association with bioresorbable beads or particles and a bioresorbable gel and/or gel-forming substance, wherein the cells and/or suitable progenitor cells thereof in association with the bioresorbable beads or particles are uniformly mixed in the bioresorbable gel and/or gel-forming substance before administration to the subject.

137. The method of claim 136, wherein said cells and/or suitable progenitor cells thereof in association with said bioresorbable beads or particles and a gel and/or gel-forming substance, are administered at said site by entrapping the cells and/or suitable progenitor cells thereof in association with said bioresorbable beads or particles and gel and/or gel-forming substance under a tissue flap or other membranous flap.

138. The method of claim 136, wherein said cells and/or progenitor cells are associated with the beads or particles by being bound thereto.

139. The method of claim 136, wherein said bioresorbable beads or particles are comprised of a biologically-based polymer(s) selected from the group consisting of gelatin and collagen, a synthetic polymer(s) selected from the group consisting of poly(glycolide), poly(lactide) and poly(lactide-co-glycolide), or a mixture of said biologically-based polymer(s) and synthetic polymer(s).

140. The method of claim 136, wherein said bioresorbable beads or particles have been functionalised or coated in a suitable cell adherence-enhancing material.

141. The method of claim 136, wherein said bioresorbable beads or particles have a diameter or dimension sized in the range of about 20 to 2500 μm.

142. The method of claim 141, wherein the average size of said bioresorbable beads or particles is about 50 to 200 μm.

143. The method of claim 136, wherein said gel and/or gel-forming substance comprises a biologically-based polymer(s) selected from the group consisting of collagen, fibrin, hyaluronan, chitosan and mixtures thereof, a synthetic polymer(s) selected from the group consisting of photopolymerizable end-capped block copolymers of poly(ethylene oxide) and an α-hydroxy acid, or a mixture of said biologically-based polymer(s) and synthetic polymer(s).

144. The method of claim 136, wherein said gel and/or gel-forming substance is further comprised of a beneficial agent(s) selected from the group consisting of growth factors, glycosaminoglycans and hydrophilic compounds.

145. The method of claim 136, wherein said gel and/or gel-forming substance includes an adhesive material(s).

146. The method of claim 136, wherein said cells and/or progenitor cells comprises chondrocytes, embryonic stem cells and/or bone marrow stromal cells.

147. The method of claims 136, wherein said cells and/or progenitor cells are chondrocytes and the diseased or damaged tissue to be treated is articular cartilage.

148. A method for augmenting tissue in a subject, said method comprising administering by injection to said subject at a site where tissue is to be augmented, cells of a type(s) normally found in the tissue to be augmented, and/or suitable progenitor cells thereof, in association with bioresorbable beads or particles and a bioresorbable gel and/or gel-forming substance, wherein the cells and/or suitable progenitor cells thereof in association with the bioresorbable beads or particles are uniformly mixed in the bioresorbable gel and/or gel-forming substance before administration to the subject.

149. The method of claim 148, wherein said cells and/or progenitor cells are associated with the beads or particles by being bound thereto.

150. The method of claim 148, wherein said bioresorbable beads or particles are comprised of a biologically-based polymer(s) selected from the group consisting of gelatin and collagen, a synthetic polymer(s) selected from the group consisting of poly(glycolide), poly(lactide) and poly(lactide-co-glycolide), or a mixture of said biologically-based polymer(s) and synthetic polymer(s).

151. The method of claim 148, wherein said bioresorbable beads or particles have been functionalised or coated in a suitable cell adherence-enhancing material.

152. The method of claim 148, wherein said bioresorbable beads or particles have a diameter or dimension sized in the range of about 20 to 2500 μm.

153. The method of claim 152, wherein the average size of said bioresorbable beads or particles is about 50 to 200 μm.

154. The method of claim 148, wherein said gel and/or gel-forming substance comprises a biologically-based polymer(s) selected from the group consisting of collagen, fibrin, hyaluronan, chitosan and mixtures thereof, a synthetic polymer(s) selected from the group consisting of photopolymerizable end-capped block copolymers of poly(ethylene oxide) and an α-hydroxy acid, or a mixture of said biologically-based polymer(s) and synthetic polymer(s).

155. The method of claim 148, wherein said gel and/or gel-forming substance is further comprised of a beneficial agent(s) selected from the group consisting of growth factors, glycosaminoglycans and hydrophilic compounds.

156. The method of claim 148, wherein said gel and/or gel-forming substance includes an adhesive material(s).

157. The method of claim 148, wherein said cells and/or progenitor cells comprises chondrocytes, embryonic stem cells and/or bone marrow stromal cells.

158. The method of claims 148, wherein said cells and/or progenitor cells are chondrocytes and the tissue to be augmented is articular cartilage.

159. A method for treating disease or damaged tissue in a subject, said method comprising the steps of:
(i) obtaining cells of a type(s) normally found in healthy tissue corresponding to said diseased or damaged tissue and/or suitable progenitor cells thereof,
(ii) expanding said cells and/or progenitor cells in the presence of bioresorbable beads or particles whereby said expanded cells and/or progenitor cells become bound to the said beads or particles, or otherwise expanding said cells and/or progenitor cells and thereafter binding said expanded cells and/or progenitor cells to bioresorbable beads or particles,
(iii) administering by injection to said subject within or under tissue at a site wherein said diseased or damaged tissue occurs, the beads or particles with said cells and/or progenitor cells bound thereto in a bioresorbable gel and/or gel-forming substance, wherein the cells and/or suitable progenitor cells thereof in association with the bioresorbable beads or particles are uniformly mixed in the bioresorbable gel and/or gel-forming substance before administration to the subject.

160. The method of claim 159, wherein said step (ii) involves expanding said cells and/or progenitor cells in the presence of bioresorbable beads or particles in a bioreactor containing a suitable culture medium, and wherein said culture medium is agitated and aerated.

161. The method of claim 160, wherein said bioreactor is a tumbler-type bioreactor equipped with internal vanes to assist in movement of the cells and/or progenitor cells, culture medium and bioresorbable beads and/or particles.

162. The method of claim 159, wherein said bioresorbable beads or particles are comprised of a biologically-based polymer(s) selected from the group consisting of gelatin and collagen, a synthetic polymer(s) selected from the group consisting of poly(glycolide), poly(lactide) and poly(lactide-co-glycolide), or a mixture of said biologically-based polymer(s) and synthetic polymer(s).

163. The method of claim 159, wherein said bioresorbable beads or particles have been functionalised or coated in a suitable cell adherence-enhancing material.

164. The method of claim 159, wherein said bioresorbable beads or particles have a diameter or dimension sized in the range of about 20 to 2500 μm.

165. The method of claim 159, wherein the average size of said bioresorbable beads or particles is about 50 to 200 μm.

166. The method of claim 159, wherein said gel and/or gel-forming substance comprises a biologically-based polymer(s) selected from the group consisting of collagen, fibrin, hyaluronan, chitosan and mixtures thereof, a synthetic polymer(s) selected from the group consisting of photopolymerizable end-capped block copolymers of poly(ethylene oxide) and an α-hydroxy acid, or a mixture of said biologically-based polymer(s) and a synthetic polymer(s).

167. The method of claim 159, wherein said gel and/or gel-forming substance is further comprised of a beneficial agent(s) selected from the group consisting of growth factors, glycosaminoglycans and hydrophilic compounds.

168. The method of claim 159, wherein said gel and/or gel-forming substance includes an adhesive material(s).

169. The method of claim 159, wherein said cells and/or progenitor cells comprise chondrocytes, embryonic stem cells and/or bone marrow stromal cells.

170. The method of claim 159, wherein said step (ii) expands the cells and/or progenitor cells 5 to 2000-fold.

171. The method of claim 170, wherein said step (ii) expands the cells and/or progenitor cells 10 to 100-fold.

172. The method of claim 159, wherein said cells and/or suitable progenitor cells are chondrocytes and the diseased or damaged tissue to be treated is articular cartilage.

173. The method of claim 159, wherein said cells and/or suitable progenitor cells thereof in association with said bioresorbable beads or particles and a gel and/or gel-forming substance, are administered at said site by entrapping the cells and/or suitable progenitor cells thereof in association with said bioresorbable beads or particles and gel and/or gel-forming substance under a tissue flap or other membranous flap.
